(19) United States
(12) Patent Application Publication
(10) Pub. No.: US 2014/0227692 A1
(43) Pub. Date: Aug. 14, 2014

(54) METHODS OF DETECTING HEREDITARY CANCER PREDISPOSITION

(71) Applicants: Deborah W. Neklason, Salt Lake City, UT (US); Randall W. Burt, Sandy, UT (US)

(72) Inventors: Deborah W. Neklason, Salt Lake City, UT (US); Randall W. Burt, Sandy, UT (US)

(21) Appl. No.: 14/131,671

(22) PCT Filed: Jul. 6, 2012
(86) PCT No.: PCT/US2012/045795

§ 371 Date: Apr. 3, 2014

Related U.S. Application Data
(60) Provisional application No. 61/505,926, filed on Jul. 8, 2011.

(51) Int. Cl. C12Q 1/68

(52) U.S. Cl. CPC C12Q 1/6886; C12Q 2531/113
USPC 435/6.11

(57) ABSTRACT

Methods for detecting hereditary cancer predisposition are disclosed.

### FEDERALLY SPONSORED RESEARCH OR DEVELOPMENT

This invention was made with government support under Grant #U24 CA078174 and #R03 CA150067 awarded by the National Institutes of Health. The Government has certain rights in the invention.

### BACKGROUND

Studies have shown that a portion of cancers of various types have a hereditary component. Risk alleles continue to be discovered. By identifying risk alleles and correlating them to their specific cancer phenotypes, diagnostic methods may be developed to provide information relevant both to assessing risk of developing cancer and selecting effective treatment options.

The MET oncogene has been associated with various steps leading to neoplasia and metastasis. It is located on chromosome 7, band 7q21-q31 and codes for a protein that has 1390 amino acids. This protein is cleaved to two subunits, an alpha and a beta chain, which are linked by a disulfide bond.

MET is expressed mainly on the surface of epithelial cells. Its c-terminal tyrosine residues are phosphorylated in response to the binding of its ligand, hepatocyte growth factor (HGF). A cascade of intracellular signals are then triggered which result in activation of MAPK and/or PI3k/Akt pathways. Aberrant activation of MET leads to increased cell proliferation, invasion, and metastasis.

MET participates in signal transduction that is thought to play a role in a variety of types of cancer. Mutations in the MET gene have been found in individuals with carcinomas of the breast, liver, lung, ovary, kidney, colon/rectum, and thyroid gland. Overexpression of MET has been associated with aggressive cancers and poor prognosis.

MET is the target for therapeutics that include small molecules (see U.S. Patent Application No. 2012/0070368, U.S. Pat. Nos. 7,880,004 and 7,919,502), antibodies (see U.S. Patent Application Nos. 2012/0082662, 2012/0082663, 2012/0076775), and splice variants of the MET protein expressed as fusion proteins (see U.S. Pat. No. 7,758,862,). These disclosures propose the use of MET signaling inhibitors in the treatment of a variety of cancers. These cancers include, but are not limited to, breast, brain, lung, kidney, gastric, head and neck, liver, ovarian, pancreas/gall bladder, prostate, thyroid, osteosarcoma, rhabdomyosarcoma, MFH/fibrosarcoma, glioblastomas/astrocytomas, melanoma, and mesothelioma and colorectal cancers.

Linkage of the 7q31 locus in a cohort of sibling pairs affected with colorectal cancer has previously been reported. MET is one of the candidate genes within this region. This gene is found to be amplified in approximately 30% of colorectal cancers, and is associated with advanced stages and poor prognosis. Additionally, missense mutations are found in 3-9% of colorectal cancers, in particular p.R970C and p.T992I based on NM_{—}000245.2, NP_{—}000236.2.

Ten percent of all colon cancers arise in a familial setting with two or more affected first degree relatives. There is a two-fold increased risk of developing colon cancer with a first degree relative that has been diagnosed with colon cancer. As mentioned above, mutations, including those in MET, have been reported in various genes present in cancerous tissues, but not all of these alleles are sufficient to predispose an individual to cancer, or contribute to the cancer phenotype at all. Although a particular mutation may be identified in somatic cancerous tissue, it is impossible to know a priori whether this particular mutation is present in the germ line. Consequently, while it is relevant that the MET gene is amplified in 30% of colon cancer tissues, this does not confirm that MET causes hereditary colon cancer or that mutations or aberrant expression are present in normal tissue.

The MET gene is also well described as a participant in the melanoma disease process. Like colorectal cancer, melanoma is known to have a genetic component in many cases. One estimate is that from 5-10% of melanomas are hereditary. Florell et al. (2005) J Clin Oncol. 23:7168-77. However, like colon cancer, it is not known whether MET variations participate in hereditary melanoma progression.

MET signaling has been linked to melanoma by observing elevated MET protein expression, elevated levels of activated (phosphorylated) MET protein, and by examining the effects of overexpressing HGF, the ligand for MET. In one study, 82.5% of uveal melanomas examined expressed activated MET protein. In addition, transgenic mice that overexpress HGF were found to be predisposed to a wide variety of carcinomas and sarcomas with cutaneous malignant melanoma being the most prevalent. Otsuka, et al. (1998) Cancer Res. 58:5157-67. This observation suggests that excessive MET signaling may lead to malignant melanoma. Still another study found that human melanoma cell lines express MET and that a c-MET-specific antagonist inhibits proliferation of these cell lines. Puri et al. (2007) Clin. Cancer Res. 13:2246-42. Furthermore, mutations were identified in the MET gene of both melanoma cell lines and tumors. Id.

While a number of mutations have been identified within the MET gene and some of them have been linked to specific types of cancer, the location of the mutation may determine what type of cancer develops. Specifically, mutant MET transgenic mouse lines with different MET mutations developed unique tumor profiles including carcinomas, sarcomas, and lymphomas. Graveel et al. 2005. Cell Cycle 4:518-20. Consequently, it is important to correlate the specific mutation in the MET gene with the disease phenotype with which it is associated.

U.S. Patent Application Publication No. 2009/0155807 discloses mutations in the MET gene that result in amino acid substitutions at positions N375, 1638, V13, V923, I316 and/or E168. The patent discloses these amino acid substitutions specifically as indicators of lung cancer. It does not, however, disclose the germline T992I mutation as disclosed herein.

U.S. Pat. No. 7,964,365 claims the use of measuring the concentration of soluble c-Met ectodomain in a biological fluid from a patient and comparing this to normal levels in order to assess risk of developing cancer. This patent does not differentiate between wild type and mutant forms of the protein or disclose the step of identifying a mutation in the MET gene.

Ma et al., (2005) Cancer Res. 65:1479-88, discloses unique activating mutations within the MET intracellular domain (R988C (defined as R990C in other reports), R998C+ T1010I (defined as T992I herein), and 51058P) and within the semaphorin (Sema) domain (E168D, L299F, S323G, and N375S). This study also discloses an alternative splice product that skips the entire juxtamembrane domain. These mutations were identified in non-small cell lung cancer cell lines and adenocarcinoma tissues. However, these mutations differ from the T992I mutation disclosed herein. Also, these mutations were identified in somatic tissue and were not shown to be germline mutations.

Schmidt et al., (1997) Nat. Genet. 16:68-73, disclose distinct missense mutations located in the tyrosine kinase domain of the MET gene in the germline of affected members of papillary renal carcinoma families. As with Ma et al. (2005), the authors of this study do not disclose the mutation claimed herein. In addition, the mutation disclosed herein has not been correlated to hereditary papillary renal carcinoma.

Wasenius et al (2005) Am J Surg Pathol 29:544-49, disclose a missense MET sequence alteration T1010I (defined as T992I herein) in thyroid carcinomas and germline of a subset of affected individuals. The authors suggest a limited role in the molecular pathogenesis of thyroid carcinoma.

### SUMMARY

The present disclosure is related to methods for detecting hereditary cancer predisposition in an individual.

In one embodiment, the method of detecting hereditary cancer predisposition in the individual comprises: obtaining a DNA sample from the individual and assaying whether the individual harbors a germline p.T992I allele at the MET gene. The presence of a germline p.T992I allele is indicative of increased risk of hereditary cancer.

In one embodiment, the p.T992I allele comprises a MET c.2975C>T allele. In another embodiment, the hereditary cancer is hereditary colon cancer. In still another embodiment, the hereditary cancer is hereditary melanoma.

In a related embodiment, the step of obtaining the DNA sample from the individual comprises obtaining a tissue sample from the individual and extracting DNA from the tissue sample. In some embodiments, the tissue sample is a blood or buccal sample.

In some embodiments, the step of assaying whether the individual harbors a germline p.T992I allele at the MET gene comprises amplifying at least a portion of the MET gene using PCR.

In a related embodiment, amplifying at least a portion of MET gene is performed in the presence of at least one nucleic acid probe, wherein the nucleic acid probe is complementary to a sequence in the MET gene that includes the codon that codes for amino acid 992. In further related embodiments, the nucleic acid probe is complementary to a MET allele that codes for a T992I mutation. In other related embodiments, the nucleic acid probe is coupled to a fluorophore.

In some embodiments comprising amplifying at least a portion of the MET gene using PCR, the method further comprises the step of sequencing the amplified portion of the MET gene.

In some embodiments, the method further comprises the step of determining whether a relative of the individual harbors a germline T992I allele at the MET gene.

Additional aspects and advantages will be apparent from the following detailed description of preferred embodiments, which proceeds with reference to the accompanying drawings.

### BRIEF DESCRIPTION OF THE DRAWINGS

FIG. 1 depicts the location of mutations identified in MET oncogene.

FIG. 2 depicts a partial pedigree of a family harboring the germline T992I allele.

### DETAILED DESCRIPTION

The present disclosure is generally related to methods of determining the predisposition of an individual to hereditary cancer. While it is well known that many cancers have a genetic component, many of the mutations that account for the increased risk remain unknown. In the case of colorectal cancer, twin studies suggest that inherited and/or familial factors contribute to 25-35% of cases. However, the highly penetrant inherited colon cancer conditions including familial adenomatous polyposis (FAP), Lynch syndrome (hereditary nonpolyposis colorectal cancer), Peutz-Jeghers, Cowden syndrome and juvenile polyposis account for only about 5% of colorectal cancers. This leaves greater than 20% without clear genetic definition.

The oncogene MET is found to be amplified in approximately 10% of colorectal cancer cases and the missense mutation T992I has been reported in tissues representing colorectal cancer, endometrial cancer, melanoma, thyroid cancer, mesothelioma, chronic lymphocytic leukemia (CLL), and acute myeloid leukemia (AML). However, the role of this mutation in hereditary cancer, which requires that a germline mutation be identified in cohorts that have abnormally high incidence of cancer, has not previously been shown.

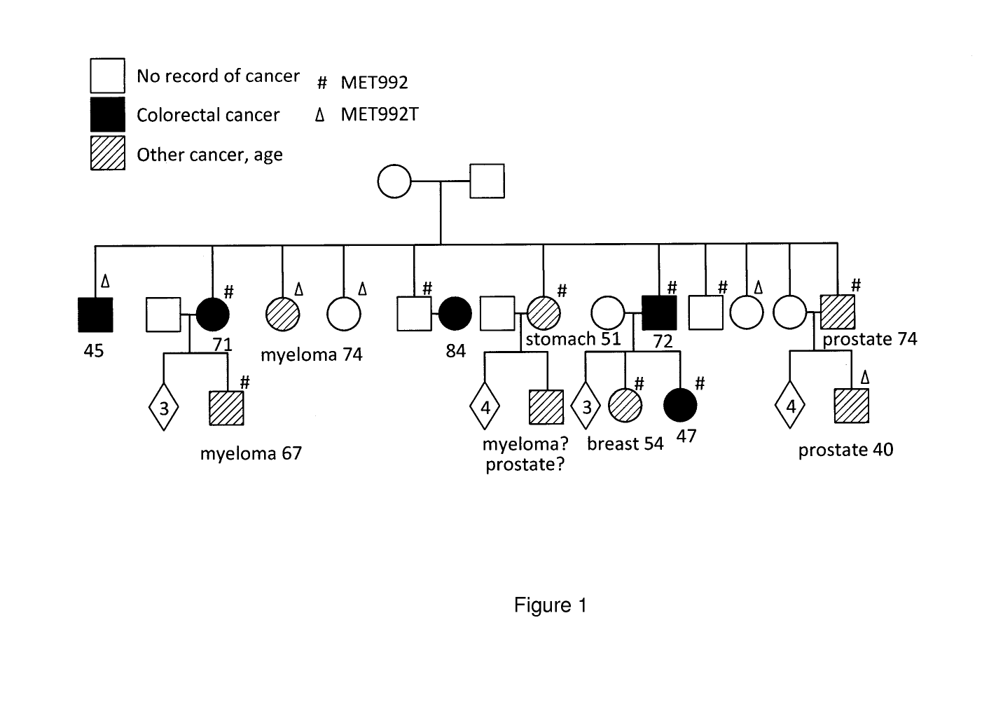

FIG. 1 depicts the MET protein with mutations indicated at both the DNA and amino acid levels. The MET protein is 1390 amino acids long (NM_{—}000245.2, NP_{—}000236.2). The pre-protein is cleaved into an alpha and a beta chain that are joined by a disulfide bond to create the extracellular receptor. The extracellular region (grey) contains a Sema domain (region of homology to semaphorins), followed by a PSI (from Plexin Semaphorins Integrins) and four IPT domains (related to immunoglobulin-like domains) and is encoded in exons 2-12 of the mRNA. The transmembrane domain (black) is encoded in exon 13. The intracellular domain (white) is encoded in exons 14-21. Serine 985 ($) down-regulates kinase activity when phosphorylated, tyrosine 1234 and 1235 (#) positively modulate enzyme activity when phosphorylated, and tyrosine 1349 and 1356 (*) recruit signal transducers when phosphorylated. Catalog of Somatic Mutations in Cancer (COSMIC) reports mutations in amino acids 130-370 of the extracellular domain, and 960-1340 of the intracellular tyrosine kinase domain. Amino acid changes responsible for Hereditary Papillary Renal Carcinoma (HPRC) are found between amino acids 1110 and 1268. Sequencing traces of the sequence mutations in the colon cancer sibling pair population are shown with their approximate location on the gene.

In one general aspect, the method comprises obtaining a DNA sample from the individual, and assaying whether the individual harbors a germline p.T992I allele within the MET gene. The presence of a germline p.T992I allele is indicative of increased risk of hereditary cancer. In certain embodiments, the p.T992I allele comprises a MET c.2975C>T allele. In certain embodiments, the hereditary cancer is hereditary colon cancer. In still other embodiments, the hereditary cancer is melanoma.

In the present disclosure, “obtaining a DNA sample from the individual” means obtaining, from any source, a sample of DNA from the individual. For example, the DNA sample may be obtained from a source such as a lab, or other third party that had previously extracted a DNA sample from the individual. In some embodiments, the DNA sample may be obtained by extracting DNA from a tissue sample from the individual. The tissue sample may be obtained directly from the individual, or it may be obtained from a third party (for example, a physician, or lab) which had previously obtained the tissue sample from the individual. It some embodiments, the tissue sample is healthy tissue to ensure that the extracted DNA is representative of the germline DNA.

In some embodiments, the tissue sample is a preserved tissue sample. In some embodiments, the tissue sample is a biopsy tissue sample. In some embodiments, the tissue sample is a blood sample. In some embodiments, the tissue sample is a buccal sample.

Methods of extracting DNA from a tissue sample are well known in the art and the skilled person would be able to determine how to extract DNA from a given tissue sample. For example, methods of extracting DNA from a tissue sample are provided in Short Protocols in Molecular Biology (2002—5th Ed.), edited by Ausubell et al., which is hereby incorporated by reference in its entirety.

In the present disclosure, “assaying whether the individual harbors a germline p.T992I allele at the MET gene” means using any known method in the art to determine whether the individual's germline DNA harbors a MET allele that codes for an isoleucine at amino acid position 992 of the translation product of the MET gene (NM_{—}000245.2, NP_{—}000236.2). The MET alleles that code for an isoleucine at amino acid position 992 often comprise a single nucleotide polymorphism (“SNP”) in the codon the codes for amino acid 992. For example, a C>T transition at the MET nucleotide equivalent to nucleotide 2975 of the MET cDNA will result in a p.T992I mutation in the MET gene product.

There are various methods known in the art for determining whether a SNP exists at a particular region of DNA, and the skilled person would choose the appropriate method for the particular SNP and the particular DNA sample provided. Several examples of methods of SNP genotyping are provided below.

SNPs may be genotyped using microarray-hybridization methods, ligation methods, and primer extension methods as described in Rapley, R. and Harbron, S. E. (2004) Molecular Analysis and Genome Discovery. Sussex, UK: Wiley, which is hereby incorporated by reference in its entirety. Furthermore, SNP genotyping may be performed using methods involving the use of a nuclease, such as the Invader assay using FLAP endonuclease. The Invader assay is described at length Olivier M. (2005) Mutat Res. 573(1-2):103-10, which is hereby incorporated by reference in its entirety. SNPs may further be detected through a single strand conformation polymorphism, as described in Costabile M., Quach A., Ferrante A. (2006) Hum Mutat. 27(12):1163-73, which is hereby incorporated by reference in its entirety.

SNPs may be detected using Polymerase Chain Reaction (“PCR”) assays. PCR-based assays are useful because they do not require large starting samples of DNA. In PCR-based assays, the region suspected of harboring a SNP is amplified using PCR techniques well known in the art.

The T992I missense mutation has been found in colorectal cancer tissues. Fumagalli et al. (2010) BMC Cancer 10:101-14, which is hereby incorporated by reference. In some embodiments, a region of the MET gene may be amplified to assay whether the individual harbors a germline p.T992I allele at the MET gene. A skilled person would be able to determine, in light of the present disclosure and the teachings of the prior art, how to choose appropriate primers and reaction conditions to successfully amplify a region of the MET gene. In some embodiments, the region of the MET gene that codes for amino acid 992 in the MET gene product is amplified.

In some embodiments, the assay is completed after the PCR product is obtained. For example, high resolution melting analysis may be performed using a fluorophore that fluoresces brightly in the presence of double stranded DNA, as described in, for example, Krypuy M, Ahmed A A, Etemadmoghadam D, et al. (2007) BMC Cancer 7:168, which is hereby incorporated by reference.

Furthermore, a PCR product may be sequenced using standard and well known sequencing methods to determine whether a SNP is present. A PCR product may also be subjected to mass spectrometry to detect SNPs, as described in Gunderson K. L., Steemers F. J., Ren H., et al. (2006) Methods Enzymol. 410:359-76, which is hereby incorporated by reference. Other methods of detecting a SNP in a PCR product are disclosed in Rapley, R. and Harbron, S. E. (2004) including, temperature gradient gel electrophoresis and denaturing high performance liquid chromatography.

In some embodiments, the assay to detect the SNP is completed while the PCR reaction is proceeding. These embodiments generally incorporate a nucleotide probe into the PCR reaction. In some embodiments, the nucleotide probe is complementary to the region that may harbor a SNP. For example, the nucleotide probe may be complementary to a sequence in the MET gene that includes the codon that codes for amino acid 992. In some embodiments, the nucleotide probe is complementary to the wild-type allele of MET. In other embodiments, the nucleotide probe is complementary to a MET gene sequence that codes for isoleucine at amino acid position 992.

In some embodiments, the nucleotide probe is coupled to a fluorophore. In some embodiments, exonuclease activity during the PCR reaction is used to release the fluorophore from the nucleotide probe. In some embodiments, a TaqMan assay is performed as described in McGuigan F. E., Ralston S. H. (2002) Psychiatr Genet. 12(3):133-6, which is hereby incorporated by reference.

In some embodiments, the genetic profile of the individual may be compared to that of a relative. For example, the presence or absence of a p.T992I allele in the individual may be compared to the genotype of a grandparent, parent or sibling. The relative's genotype may be determined, for example from population genotype databases, or using the methods already disclosed herein.

To further illustrate these embodiments, the following examples are provided. These examples are not intended to limit the scope of the claimed invention, which should be determined solely on the basis of the attached claims.

### EXAMPLES

### Example 1

### Genotyping Sibling Pairs to Identify Genetic Variants that Drive Hereditary Predisposition for Cancer

Study Population

All aspects of this study were approved by University of Utah's Institutional Review Board for human subject research. Research participants were consented to participate in a study of genetic factors leading to colon cancer risk.

Ascertainment and Collection of Colon Cancer Sibling Pairs.

Subjects were enrolled in the Inheritance of Colon Cancer: a Sibling Pair Study following review and approval by the Institutional Review Boards of the 11 participating centers. Informed consent was obtained from all research participants. Kindreds were ascertained from Cancer Genetics Network members, population-based county-wide or state-wide cancer registries, clinical referral, and direct-to-patient marketing. Each family had at least two individuals diagnosed with colorectal adenocarcinoma or a polyp with high-grade dysplasia (also reported as carcinoma in situ, which is synonymous with the previous term) of ages >20 years. Diagnosis was confirmed by pathology reports in most cases or cancer registry data. The sibling pair population was composed of 169 subjects from 77 families with 148 affected with colorectal cancer and 21 unaffected. The population is 86% Caucasian, 5% Black, 1% Native American, and 8% other.

Scanning and Sequencing of MET

Primers were designed using Primer 3 to PCR amplify each of the coding exons of MET. Amplicons were generated 250-500 bases in length using LC Green dye mastermix (Idaho Technologies), for optimal high resolution melt curve analysis (HRM) using a LightScanner (Idaho Technologies, Salt Lake City, Utah). Samples demonstrating alternative melt curves were sequenced using Applied Biosystems 3730XL capillary sequencer. Sequence traces were compiled and aligned using Sequencer™ DNA Sequence Assembly Software, version 4.10.1 build 5828 (Gene Codes Corp, Ann Arbor, Mich., USA).

Results

The coding SNPs identified and their frequencies are listed in Table 1. Three nonsynonymous changes were identified in the germline DNA of affected individuals and not recognized with a population frequency in dbSNP build 132 (www.ncbi.nlm.nih.gov/projects/SNP).

TABLE 1
Germline variants identified in MET coding sequence

| Exon | SNP* | Change* | # samples | CRC sibling frequencies | dbSNP allele frequencies |
|---|---|---|---|---|---|
| 2 | rs11762213 | synonymous | 160 | 89% GG, 11% GA | 89% GG 11% GA |
| 2 | c.577C>T | synonymous | 169 | 99% CC, 1% CT | Not reported |
| 2 | c.593G>A | V136I | 169 | 99.4% GG 0.6% GT | Not reported |
| 2 | rs35775721 | synonymous | 165 | 88% CC 12% CT | 97% CC 3% CT |
| 2 | rs55985569 | E168D | 165 | 99.4% GG 0.6% GT | 99.5% GG 0.5% GT^{#} |
| 2 | rs35776110 | A320V | 169 | 100% CC | 97% CC 3% CT |
| 2 | rs77523018 | M362T | 169 | 98% TT 2% CT | 98% TT 2% CT |
| 2 | rs33917957 | N375S | 169 | 100% AA | 97% AA 3% AG |
| 7 | rs13223756 | synonymous | 161 | 75% AA 25% AG | 67% AA 33% AG |
| 14 | rs56391007 | T992I | 163 | 95.7% CC 4.3% CT | 99.3% CC 0.7% CT^{#} |
| 20 | rs41736 | synonymous | 152 | 28% CC 54% GA 19% TT | 37% CC 45% GA 18% TT |
| 21 | rs2023748 | synonymous | 157 | 26% GG 54% GA20% AA | 37% GG 45% GA 18% AA |
| 21 | rs41737 | synonymous | 157 | 26% GG 54% GA20% AA | 37% GG 45% GA 18% AA |

*for variants without a SNP identification number in dbSNP, the nucleotide change is noted with reference to NM_000245.2 with nucleotide 1 referring to A of the AUG initiation codon and amino acid change is noted with reference to NP_000236.2.
^{#}Frequency from 1000 Genomes in dbSNP.

Two individuals had a single change in the extracellular domain of the MET receptor, p.V136I and p.E168D, however their affected siblings did not harbor the change and the changes are not predicted to disrupt protein function with both Polyphen and SIFT analysis tools. Seven of the 148 affected individuals (4.7%), and none of the unaffected siblings, have the c.2975C>T germline change which resides in the tyrosine kinase domain of MET. The seven individuals represent three affected sibling pairs and one affected individual with an unaffected sibling. The threonine amino acid at position 992 is completely invariant across vertebrates to zebrafish. This mutation has been previously reported in 2.5% of colon tumors, but not the general population (Fumagalli, D. et al. BMC Cancer 10, 101 (2010)). It is reported in dbSNP at 0.7% based on low coverage sequence of ˜600 individuals from the 1000 Genomes project (4 individuals). There are no clear pathologic characteristics of the individuals with T992I germline mutation (Table 2). The average age of onset is 61.9 years with a range of 44 to 75 years whereas the average age of the study population is 58.7 years (range 28-91).

TABLE 2
Cancer cases with MET mutations

| | | | | | | Relative |
|---|---|---|---|---|---|---|
| | | | | | | copy |
| | | | | | | number |
| | | | | Grade - | | c.2975T |
| Sam- | Muta- | | | differen- | Colonic | (Tumor/ |
| ple* | tion | Age | Stage | tiation | location | Normal) |
| sib1 | V136I | 87 | 3 | moderately | transverse | ND |
| | | | | well | | |
| sib2 | E168D | 71 | 3 | moderately | splenic | ND |
| | | | | well | flexure | |
| sib3-A | T992I | 52 | 2 | well | rectosigmoid | 1.60 |
| sib4-A | T992I | 62 | 1 | moderately | ascending | 4.32 |
| | | | | well | | |
| sib5 | T992I | 44 | 3 | poorly | rectosigmoid | 1.84 |
| sib6-B | T992I | 66 | 1 | not | descending | 1.79 |
| | | | | reported | | |
| sib7-B | T992I | 66 | 1 | not | descending | 0.07 |
| | | | | reported | | |
| sib8-C | T992I | 68 | 2 | moderately | cecum | 0.31 |
| | | | | well | | |
| sib9-C | T992I | 75 | 1 | moderately | descending | 0.38 |
| | | | | well | | |
| hr10-D | T992I | 71 | 4 | moderate | cecum | 0.06 |
| hr11 | T992I | 60 | 1 | moderate | splenic | 0.10 |
| | | | | | flexure | |
| hr12 | T992I | 51 | 2 | moderate | descending | 0.34 |
| hr13 | T992I | 77 | 2 | well | sigmoid | 0.05 |
| hr14 | T992I | 89 | 2 | moderate | rectum | 2.55 |
| hr15 | T992I | 35 | 2 | moderate | transverse | ND |
| hr16-D | T992I | 47 | 4 | moderate | cecum | ND |
| | | | | well | | |
| hr17 | T992I | 68 | 3 | moderate | sigmoid | 0.05 |
| | | | | to focally | | |
| | | | | poor | | |
| hr18 | T992I | 50 | 4 | moderate | rectum | 0.11 |

*sib indicates patient from sibling pair cohort, hr indicates patient from high risk cohort, -letter indicates same family.
Not done (ND).

Because each of the families is of Caucasian descent, we were interested in determining if there was evidence of a founder mutation. Using polymorphic markers surrounding the MET locus (D7S2418, D7S486, D7S648), the haplotype segregating with the mutation in each of the 4 families was identified and is distinct. This suggests that this mutation arose independently.

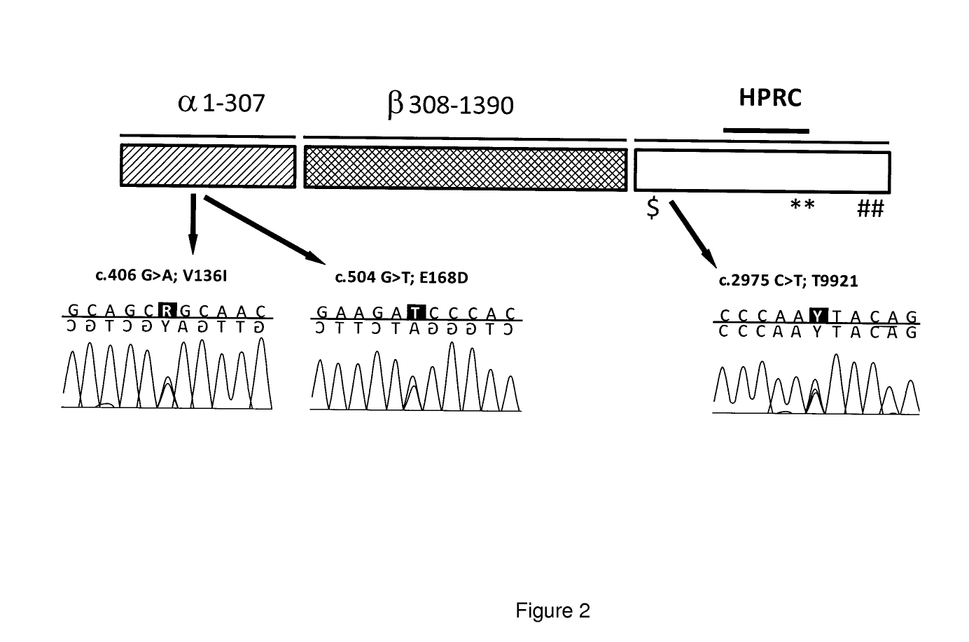

Through our initial screening of high risk colorectal, we identified two individuals that had both developed colon cancer and harbored the p.T992I allele. FIG. 2 shows a partial pedigree one of the families which is trimmed to show the top generation. The mutation status and whether the individual developed cancer are noted. We found that 23 of 80 family members harbor the p.T992I allele. The p.T992I allele segregates with three of four colon cancers. Overall, the p.T992I allele segregates with 7 of 10 cancers. It is notable that cancers other than colorectal, including two melanomas, are present in the family. Data presented below suggests that there might be a link between the p.T992I allele and melanoma. One family member suffered from both melanoma and prostate cancer. This individual has not been genotyped, and is at 50% risk of harboring the mutation.

### Example 2

### Confirming MET p.T992I Mutation as a Germline Cancer Predisposition SNP

Study Population

All aspects of this study were approved by University of Utah's Institutional Review Board for human subject research. Research participants were consented to participate in a study of genetic factors leading to colon cancer risk.

A high risk population, defined as diagnosis ≦50 years (n=130) or two first degree relatives with colorectal cancer (n=166) or both (n=3), was used for confirmation of the MET p.T992I mutation. This population has been previously described (Kerber, R. A., Neklason, D. W., Samowitz, W. S. & Burt, R. W (2005) Fam Cancer 4, 239-244) which is hereby incorporated by reference in its entirety. Diagnosis was confirmed with pathology report or state cancer registry data. Known syndromes were excluded through medical record review and molecular analysis.

MET T992I-Specific TaqMan Assay

Colon tumor DNA, micro-dissected and extracted from formalin fixed paraffin embedded (FFPE) blocks, was assayed for the presence of the MET c.2975C>T mutation (NM_{—}000245.2, NP_{—}000236.2) using ABI probe assay for rs56391007 and TaqMan Genotyping Master Mix according to manufacturers specifications. The major C allele was labeled with VIC (517 nm emission) and the minor T allele was labeled with 6FAM (554 nm emission). Products that peaked before 35 cycles on BioRad CFX96 Real-time PCR machine with a signal of >500 RFU on the respective VIC or FAP channel were called for that allele. DNA from patient-matched normal tissue (blood when available, otherwise FFPE micro-dissected normal) was examined when the mutation was identified in tumors.

Results

We found that nine of 299 tumors (3.0%) harbored the change (one from diagnosis ≦50 years (0.8%); seven from two first-degree relatives (4.2%), and one with both risk factors (33.3%)). DNA from normal tissue was available from seven of the nine cases and the mutation was present in all seven suggesting it was germline in origin. Interestingly, two of the nine are a second-degree relative pair from a large family with excess colon cancer; one ascertained as a first degree relative pair and the one ascertained as a parent-child pair diagnosed ≦50 years.

### Example 3

### Risk on Noncolorectal Cancer in Families Harboring the T992I Mutation

We obtained DNA samples from large cohort of families which demonstrated statistical excess and clustering of all cancers from a state cancer database. We refer to this study as the “Cancer Risk In Families (CRIF)” study. We tested the full set to see if the MET T992I mutation was in excess in any type of cancer other than colorectal cancer.

Results

Overall we observed a slight increase in the p.T992I allele in this cohort of 846 samples (1.8% versus 0.7% reported in 1000 genomes data). Table 3 illustrates the percent of the subjects tested that harbored the germline p.T992I and who developed the indicated type of cancer. While individuals with colorectal cancers did not demonstrate an excess of the p.T992I allele, this may be because it is mostly seen in first degree relative pairs of colorectal cancers. In support of this interpretation, Neklason et al. (2011) BMC Cancer 11:424-30, which is hereby incorporated by reference in its entirety, observed the germline p.T992I allele at a frequency of 1% in subjects that developed colorectal cancer before age 50 and who did not have a first degree relative with colorectal cancer. In contrast, subjects that had developed colon cancer and who had first degree relatives that had developed colorectal cancer were found to have a germline p.T992I mutation in the MET oncogene at a frequency of about 5%. Id.

TABLE 3
Presence of MET T992I mutation Cancer Risk in Families Cohort

| Cancer site | # tested | # positive (%) | |
|---|---|---|---|
| Prostate | 267 | 5 | (1.9%) |
| Breast | 206 | 2 | (0.97%) |
| Colorectal | 98 | 1 | (1.0%) |
| Endometrium + | 74 | 2 | (2.7%) |
| Ovary | | | |
| Melanoma | 44 | 3 | (6.8%)* |
| Thyroid | 50 | 0 | |
| Leukemia + | 29 | 0 | |
| Lymphoma | | | |
| Sarcomas | 7 | 1 | (14%) |
| Brain CNS | 5 | 1 | (20%) |
| Others | 66 | 0 | |
| Total | 846 | 15 | (1.8%) |

The three melanoma cases (6.8%) are particularly relevant in light of the two melanomas observed in the pedigree shown in FIG. 2. Progression of melanoma has some similarities to colorectal cancer in that they arise from a precursor lesion, colonic adenoma for colorectal cancer and nevi for melanoma. Adenomas and nevi are not considered cancerous until they take on an invasive behavior. Overactivation of the MET oncogene, through duplication or activating mutations such as the p.T992I allele, may enhance this process.

In response to the data above, we screened a set of 88 familial melanoma cases for MET T992I. None tested positive for this mutation. We suspect that greater sample numbers are needed to confirm an association between melanoma and MET T992I. Different cohorts that could be enriched for the MET T992I mutation, such as the CRIF cohort, could also be required to substantiate our hypothesis.

### Example 4

### Determining Whether Allelic Imbalance is Necessary for the p.T992I Mutation to Confer Cancer Predisposition

Because amplification of the MET gene occurs in an estimated 30% of colon cancers, we sought to determine whether the p.T992I allele was preferentially amplified in colon tumors. The Real Time quantitative PCR data from the TaqMan assay in Example 2 were used in this determination.

Allelic imbalance was evaluated using cycle-time (CT) data generated by real-time quantitative PCR with the TaqMan assay. Where ΔCT=cycle time of VIC (c.2975C, wild type allele)—cycle time of FAM (c.2975T, mutant allele), with the fold excess mutant alleles=2(ΔCT tumor−ΔCT normal).

Results

Five of the 13 cases examined showed an excess of the mutant c.2975T allele over the wild type allele (Table 2). However, overexpression of the mutant allele did not occur in the remaining 8 cases. This observation suggests that, in the presence of this mutation, additional amplification of the gene is not required for establishment of cancer.

### Example 5

### Coexistence of p.T992I with Mutation of Other Deleterious Colon Cancer Genes

We hypothesized that a germline MET T992I may not exist or, alternatively, may cause severe disease, in the presence of a germline mutation that initiated the adenoma process. In order to address this question, we examined a cohort of samples that are part of the Huntsman Cancer Institute's Hereditary Gastrointestinal Cancer Registry collected from subjects that have either clinical or genetic diagnosis of one of the known colon cancer syndromes. We also hypothesized that this mutation may be responsible for development of cancer in a subset of individuals with a high risk cancer phenotype where no mutation could be identified.

Results

Table 4 illustrates the number of subjects that were identified as having the p.T992I allele and either tested positive for an allele known to cause a colon cancer syndrome or were clinically diagnosed with one of these syndromes. We observed the presence of the T992I mutation in one FAP patient out of 312 that tested positive for the mutation in the APC gene known to cause FAP. At reported population frequencies, we would have expected to detect two such subjects. We anticipate that a larger population will be required to confirm our hypothesis that MET T992I is deleterious in the background of a germline colon cancer (adenoma) initiating mutation.

Table 4 also illustrates our analysis of the frequency of the T992I mutation in a subset of individuals where no known genetic mutation was identified but their family history and/or clinical presentation suggested a genetic predisposition. We identified the MET T992I in 4 of 159 (2.5%) of these individuals. This group included individuals with clinical features found in FAP and Cowden Syndrome and individuals with a strong history of colorectal cancer (family history or young age of onset) but no features of the known colon cancer syndromes. This observation suggests that the T992I mutation accounts for a very small fraction of individuals with clinical diagnoses of known colon cancer syndromes but no known genetic mutation. However, a larger sample size will be needed to confirm this conclusion.

TABLE 4
Presence of MET T992I mutation in HGCR cohort

| | | | |
|---|---|---|---|
| Syndrome | Gene | # tested | # positive (%) |

KNOWN GENETIC DIAGNOSIS SYNDROME

| | | | |
|---|---|---|---|
| AFAP | APC | 187 | 0 |
| FAP | APC | 77 | 1 (1.3%) |
| Lynch | MLH1, MSH2, MSH6, | 32 | 0 |
| | PMS2 | | |
| COWDEN | PTEN | 6 | 0 |
| Juvenile Polyposis | BMPR1A, SMAD4 | 5 | 0 |
| MAP | MUTYH | 2 | 0 |
| Peutz-Jeghers | STK11 | 3 | 0 |

CLINICAL DIAGNOSIS - GENE MUTATION UNKOWN

| | | | |
|---|---|---|---|
| FAP | Clinical diagnosis | 44 | 2 (4.5%) |
| COWDEN | Clinical diagnosis | 6 | 1 (16.6%) |
| High-risk, no | — | 109 | 1 (0.9%) |
| mutation | | | |

*The high-risk is individuals where a genetic predisposition is suspected including polyp formers, members of high-risk kindreds, and diagnosis of colorectal cancer under age 50.

Discussion

Ten percent of all colon cancers arise in a familial setting with 2 or more affected first degree relatives. There is a 2-fold increase of developing colon cancer with a first degree relative. There is, however, a gap in our knowledge of moderate risk genetic variants that drive this hereditary predisposition. We have examined the MET gene for germline changes that could explain colon cancer occurrence. A germline mutation in the MET gene, p.T992I, was identified in ˜4.5% of colon cancers arising in first-degree relative pairs from two separate cohorts. The mutation was observed in less than 1% of colon cancer cases that occurred ≦50 years, suggesting that it does not promote young colon cancers. There is a report of a single normal individual, as well as two melanoma and one endometrial cancer cases harboring this germline mutation and we hypothesize that this normal individual may intersect the at-risk population. MET T992I is also reported in the germline DNA of 4% of thyroid cancers.

It has been proposed that the p.T992I mutation may inhibit phosphorylation of Ser985 which, when phosphorylated, corresponds with reduced MET signaling (FIG. 1). Over expression of MET p.T992I in cultured cells results in gain of multiple metastatic behaviors including changes in cell morphology, adhesion, motility, migration and anchorage-independent growth. It, however, does not make Ba/F3 cells proliferate in an interleukin-3 independent way, suggesting that it is not a proliferative factor.

Without being bound by theory, we propose a model whereby the T992I mutation functions as a progression factor and not an initiation factor in the canonical colon cancer model. We hypothesize that an adenoma is initiated through defects in the canonical APC pathway, then the adenoma acquires other proliferative mutations, and in the presence of an underlying MET T992I mutation, is then able to move beyond the mucosal layer to become invasive colon cancer. This hypothesis is supported by the following observations. The individuals we identified with the MET p.T992I germline mutation do not have the hallmarks of inherited mutations in initiating factors such as multiple adenoma formation (APC gene) or microsatellite instability (mismatch repair genes). Additionally, colorectal cancer diagnosis under age 50 is infrequent. In fact, the chromosome 7q genetic locus is associated in affected relative pair studies when colorectal cancers are included and adenomas are excluded. The average of colorectal cancer diagnosis in the general population is ˜71 years, and it is estimated that 10 years are needed for a small polyp to progress to invasive colorectal cancer. A model of rapid progression of polyp to cancer in the presence of MET p.T992I is supported in that individuals with the p.T992I allele are diagnosed with colorectal cancer at an average age of 63 years. This would be when the general population, on average, is developing adenomas that will progress to cancer. This mutation also occurs in a variety of cancers including colon, melanoma, endometrial, thyroid, and mesothelioma with germline confirmation in colon, thyroid, uterine, and melanoma suggesting that it is not a tissue-specific mechanism. As discussed above, data presented in Table 3 herein support a role for this mutation in melanoma progression. MET protein expression is found in approximately 15% of precancerous nevi, and increases to 85% of melanomas correlating with advancing disease.

We estimate that this specific mutation is responsible for 0.45% of all colon cancers. This accounts for a substantial number of individuals considering the high prevalence of colon cancer. By offering germline genetic testing for the MET p.T992I allele, especially in a setting of family history, individuals who would benefit by frequent and early colon cancer screening can be identified so that adenomas can be removed before they become cancer. Likewise, this test may also be useful to identify individuals who could benefit from early screening for melanoma. Specific inhibitors of the MET protein are in human clinical trials and may have a specific utility in preventing the metastasis of early stage cancers in individuals with the MET T992I mutation.

It will be obvious to those having skill in the art that many changes may be made to the details of the above-described embodiments without departing from the underlying principles of the invention. The scope of the present invention should, therefore, be determined only by the following claims:

1. A method of detecting hereditary cancer predisposition in an individual comprising:
obtaining a DNA sample from the individual, and
assaying whether the individual harbors a germline p.T992I allele at the MET gene, wherein the presence of a germline p.T992I allele is indicative of increased risk of hereditary cancer.

2. The method of claim 1, wherein the hereditary cancer is hereditary colon cancer.

3. The method of claim 1, wherein the hereditary cancer is hereditary melanoma.

4. The method of claim 1, wherein the step of obtaining the DNA sample from the individual comprises obtaining a tissue sample from the individual and extracting DNA from the tissue sample.

5. The method of claim 4, wherein the tissue sample is a blood sample.

6. The method of claim 4, wherein the tissue sample is a buccal sample.

7. The method of claim 1, wherein the step of assaying whether the individual harbors a germline p.T992I allele at the MET gene comprises amplifying at least a portion of the MET gene using PCR.

8. The method of claim 7, wherein amplifying at least a portion of MET gene is performed in the presence of at least one nucleic acid probe, wherein the nucleic acid probe is complementary to a sequence in the MET gene that includes the codon that codes for amino acid 992.

9. The method of claim 8, wherein the nucleic acid probe is complementary to a MET allele that codes for a p.T992I mutation.

10. The method of claim 9, wherein the nucleic acid probe is coupled to a fluorophore.

11. The method of claim 7, further comprising the step of sequencing the amplified portion of the MET gene.
